(19) United States
(12) Patent Application Publication
(10) Pub. No.: US 2019/0328559 A1
(43) Pub. Date: Oct. 31, 2019

(54) METHODS AND APPARATUS FOR ENHANCED FLOW STENT DEVICE

(71) Applicant: High Desert Radiology, P.C., Kingman, AZ (US)

(72) Inventor: Christopher A. Porter, IV, Kingman, AZ (US)

(21) Appl. No.: 15/962,136

(22) Filed: Apr. 25, 2018

(51) Int. Cl. A61F 2/915; A61F 2/07

(52) U.S. Cl. CPC A61F 2/915; A61F 2/958; A61F 2002/91558; A61F 2/07

(57) ABSTRACT

Methods and apparatus for a stent device according to various aspects of the present technology include a deployable scaffold matrix coupled to an internal membrane bonded or attached along an inner surface of the scaffold matrix to form a lumen. A method of bonding or attaching the internal membrane to an internal surface of the external scaffold in a helical pattern will create a helical pattern along the lumen to encourage vortical flow through the lumen.

### BACKGROUND OF THE TECHNOLOGY

Deployable stents are commonly used in blood vessels to treat various types of intra-vascular conditions such as coronary heart disease, aneurysm, and peripheral arterial disease (PAD) among others. For example, coronary heart disease may be treated using intracoronary stents, in combination with percutaneous transluminal coronary angioplasty. The stents are used to maintain an increased coronary lumen diameter, and aid in treatment of dissections. In addition, covered stents can decrease the incidence of restenosis by diminishing the incidence of intimal hyperplasia caused, at least in part, by disturbed blood flow and turbulence at the site of the stent, as well as low wall shear stresses. This issue may be particularly associated with stents used in smaller diameter vessels. Accordingly, increasing laminar flow through the lumen of the stent device may lead to reductions in in-stent stenosis and aid in creating increased downstream flow.

### SUMMARY OF THE TECHNOLOGY

Methods and apparatus for a stent device according to various aspects of the present technology include a deployable scaffold matrix coupled to an internal membrane bonded or attached along an inner surface of the scaffold matrix to form a lumen. A method of bonding or attaching the internal membrane to an internal surface of the external scaffold in a helical pattern will create a helical pattern along the lumen to encourage vortical flow through the lumen.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a perspective view of a stent device in accordance with an exemplary embodiment of the present technology;

FIG. 2 representatively illustrates an end view through a lumen of the stent device in accordance with an exemplary embodiment of the present technology;

FIG. 3A representatively illustrates a top view of a flexible membrane in accordance with an exemplary embodiment of the present technology;

FIG. 3B representatively illustrates an end view of the flexible membrane in accordance with an exemplary embodiment of the present technology;

FIG. 4 representatively illustrates a cross-sectional view along line 4-4 of FIG. 1 in accordance with an exemplary embodiment of the present technology;

FIG. 5 representatively illustrates the flexible membrane shown in FIG. 4 in a flexed position in accordance with an exemplary embodiment of the present technology;

FIG. 6 representatively illustrates bonding locations between the flexible membrane and a stent scaffold in accordance with an exemplary embodiment of the present technology;

FIG. 7 representatively illustrates a perspective view of a stent device having an alternative bonding method in accordance with an exemplary embodiment of the present technology; and

FIG. 8 representatively illustrates a cross-sectional view along line 8-8 of FIG. 7 in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of hardware components configured to perform the specified functions and achieve the various results. For example, the present technology may be used in conjunction with various materials, needles, wires, injectable devices, dilators, ports, and the like, which may carry out a variety functions. In addition, the present technology may be practiced in conjunction with any number of endovascular applications, and the system described is merely one exemplary application for the technology.

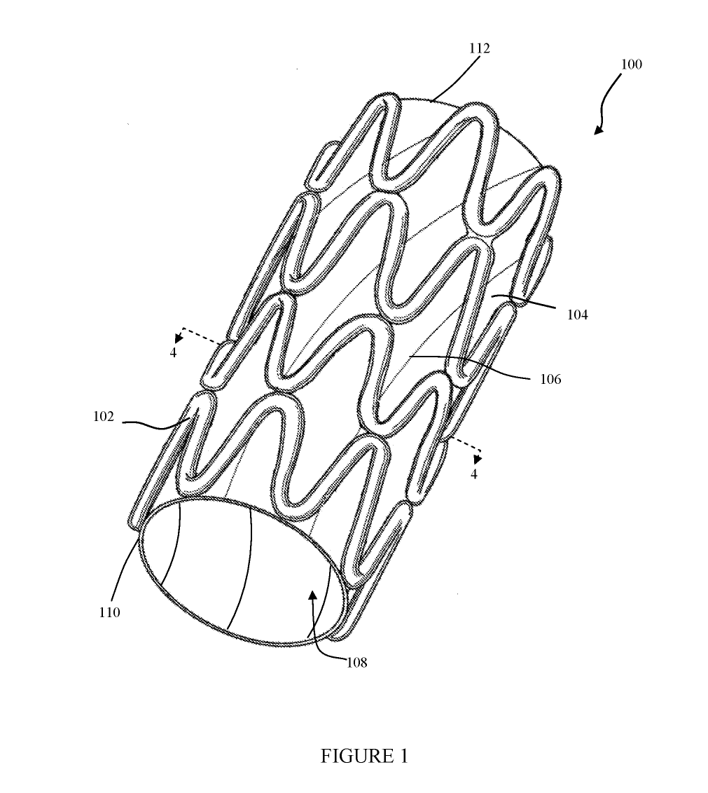

Referring now to FIG. 1 a stent device 100 according to various aspects of the present technology may comprise a membrane 104 coupled to an inner surface of a stent scaffold 102 to form a lumen 108 having a first end 110 and a second end 112. An inner surface of the lumen 108 may comprise a surface configured to encourage vortical flow between the first and second ends 110, 112 of the lumen 108 when positioned within an anatomic vessel.

The stent scaffold 102 provides a rigid or semi-rigid frame that is coupled to the membrane 104 to help form the lumen 108. The stent scaffold 102 may comprise any suitable device such as a metal or synthetic wire frame or mesh formed in a generally tubular shape. For example, in one embodiment, the stent scaffold 102 may comprise an expandable metal alloy that is configured to expand outwardly from a constrained first position to a second deployed position having a larger diameter than in the first position. In the constrained position, the stent scaffold 102 may be held at a first diameter and positioned within a constraining sleeve/sheath such as those commonly used in endovascular procedures. Once positioned in a target vessel, the stent scaffold 102 may be deployed and allowed to expand outwardly from a central axis of the stent scaffold 102 to the inner wall of the target vessel.

The stent scaffold 102 may be expanded outward according to any suitable method. For example, a balloon may be inserted into, or otherwise positioned within, the center of the stent scaffold 102 and insufflated to expand the stent scaffold 102 outward to its full working diameter. Alternatively, the stent scaffold 102 may be self-expanding such that once it is no longer constrained by the constraining sheath, the stent scaffold 102 may expand to a larger working diameter without the need for an external force.

The stent scaffold 102 may be expandable between any two desired diameters according to a desired application or target vessel. For example, coronary arteries may require a stent scaffold 102 having a deployed diameter of at least three millimeters while peripheral arteries such as those found in the legs may be less than two millimeters in diameter and require an appropriately smaller sized stent scaffold 102 when deployed. The stent scaffold could be used in larger vessels requiring a stent scaffold of suitable diameter. Similarly, the stent scaffold 102 may comprise any suitable length between about three millimeters and about twenty centimeters that may be determined according to a desired application or need.

The stent scaffold 102 may comprise any suitable material suitable for use within a living organism. For example, in one embodiment, the stent scaffold 102 may comprise nitinol. In alternative embodiments, the stent scaffold 102 may comprise stainless steel or a bioabsorbable material such as magnesium, zinc, or a polymer.

The membrane 104 provides a surface along the inner surface of the stent scaffold 102 to allow fluid flow through the lumen 108. The membrane 104 may comprise any suitable device or system for providing a fluid flow path through the inner portion of the stent scaffold 102 and the membrane 104 may comprise any material suitable for use suitable for use within a living organism such as polytetrafluoroethylene (PTFE), nanofibrous PTFE, or other like polymers or materials.

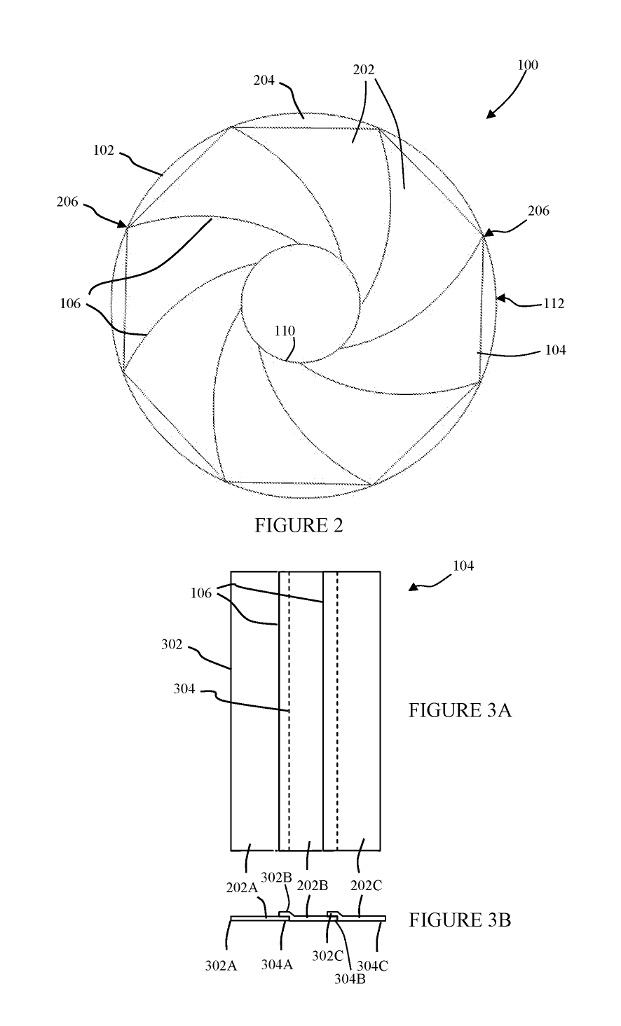

Referring now to FIGS. 1 and 2, an inner surface of the lumen 108 may comprise a helical pattern that extends from the first end 110 to the second end 112 of the lumen 108. The helical pattern creates a “rifling” affect along the inner surface of the lumen 108 that is configured to increase vortical (rotational) flow of a fluid about a central axis of the lumen 108 as the fluid passes through the stent device 100. Increased vortical flow of the fluid may improve the overall laminar flow of the fluid through the stent device 100 thereby reducing localized pressure differentials, turbulence, and other factors that may work to impede fluid flow in a desired direction though the lumen 108.

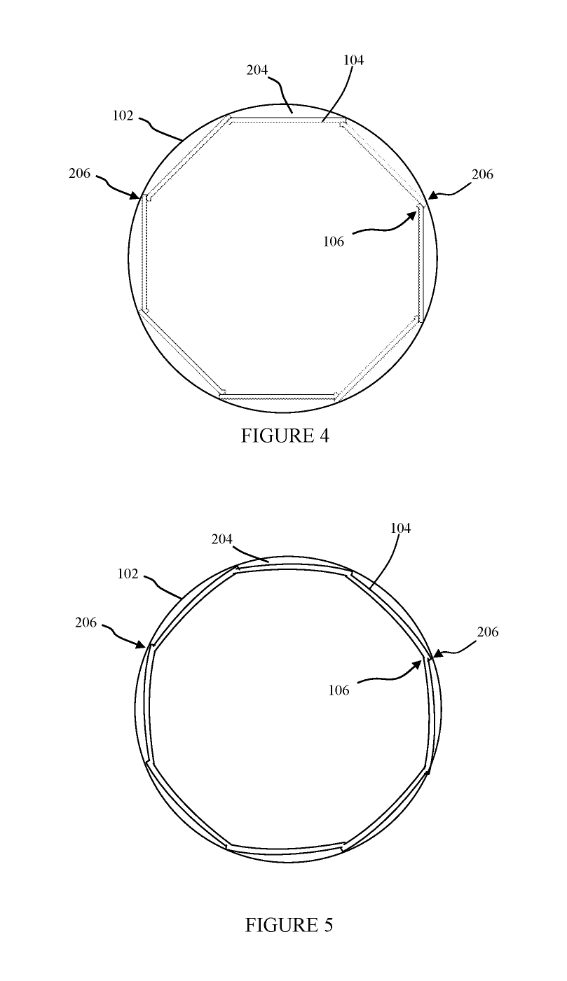

In one embodiment, the membrane 104 may comprise a plurality of membrane components 202 coupled together and arranged along the inner surface of the stent scaffold 102 in a longitudinal-linear-helical manner. The plurality of membrane components 202 may be coupled, or otherwise bonded together to form a plurality of ridges 106 that extend along the entire length of the lumen 108 between the first and second ends 110, 112. In one embodiment, and referring now to FIGS. 3A, 3B, and 4, each membrane component 202 may comprise a first side edge 302 and a second side edge 304. The first and second side edges 302, 304 of each membrane component 202 may be coupled to an adjacent membrane component 202. For example, the second side edge 304A of a first membrane component 202A may be coupled to the first side edge 304B of an immediately adjacent second membrane component 202B. Similarly, the second side edge 304B of the second membrane component 202B may be coupled to the first side edge 302C of an immediately adjacent third membrane component 202C. This pattern may repeat until each membrane component 202 is coupled to another membrane component 202 forming a generally tubular shape.

The first and second side edges 302, 304 may be coupled together by any suitable method. In one embodiment, the first and second side edges 302, 304 may be bonded together in an overlapping fashion. In an alternative embodiment, the first and second side edges 302, 304 may be bonded in an edge-to-edge manner without any overlap. Bonding to the external matrix in a helical pattern may be achieved mechanically, thermally, chemically, or adhesively.

At each location where one membrane component 202 is coupled to another membrane component 202 a ridge 106 may be formed. The assembled tubular membrane 104 may then be coupled to the stent scaffold 102 such that the ridges 106 do not run directly parallel along the length of the lumen 108. For example, referring now to FIG. 6, the membrane 104 bonding pattern may be rotated to a predetermined angle relative to the stent scaffold 102 such that the ridges are angled along the length of the stent scaffold 102. Accordingly, when opposing sides 602, 604 of the stent scaffold 102 and membrane 104 are joined together the ridges 106 will form a helical pattern that proceeds along the entire length of stent device 100 as shown in FIG. 2. The helical paths the ridges 106 take along the length of the lumen 108 create the “rifling effect” used to encourage vortical fluid flow.

Alternatively, each membrane component 202 may be individually coupled to the inner surface of the tubular stent scaffold 102 in a longitudinal-linear-helical fashion. For example, adjacent membrane components 202 may overlap and be bonded together along their edges similar to that shown in FIG. 3A. In this embodiment, the ridges 106 formed between adjacent membrane components are formed by the joint formed between adjacent membrane components 202 and proceed along the length of the lumen 108 in a helical manner as described above.

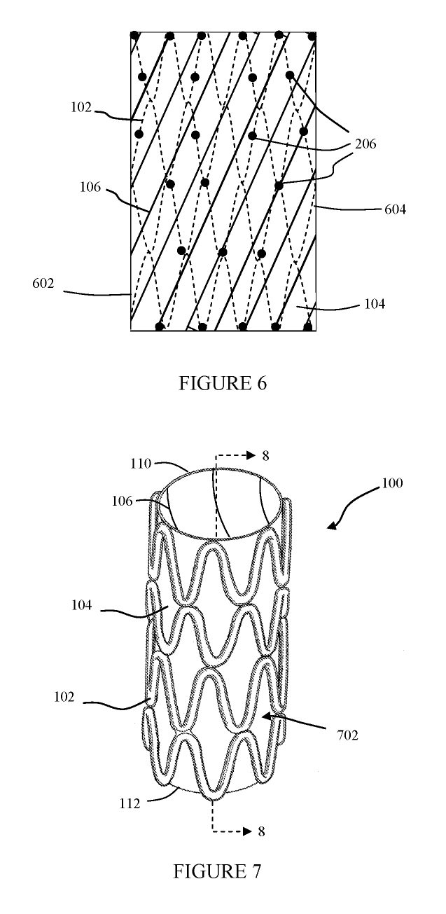

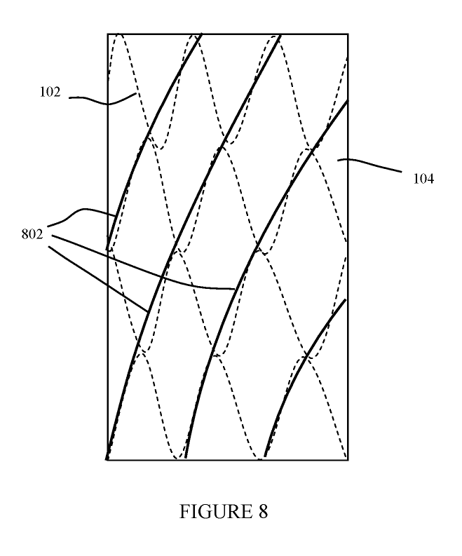

In another embodiment, the membrane 104 may comprise a circumferentially contiguous single membrane component bonded in a helical pattern to the inner surface of the stent scaffold 102. For example, and referring now to FIGS. 7 and 8, an outer surface 702 of the membrane 104 may be coupled to the inner surface of the stent scaffold 102 along a plurality of bond lines 802 arranged in a helical manner between the first and second ends 110, 112. The bond lines 802 between the membrane 104 and the stent scaffold 102 create the ridges 106 that form the helical pattern along the length of the lumen to encourage vortical flow as described above.

The membrane 104 may be coupled to the stent scaffold 102 in any suitable manner. In one embodiment, the entire inner surface of the stent scaffold 102 may be bonded to the membrane 104. In an alternative embodiment, and referring now to FIGS. 2 and 4-6, the membrane 104 may only be bonded or otherwise coupled to the inner surface of the stent scaffold 102 at a plurality of point locations 206. Alternatively, and referring to FIG. 8, the membrane 104 may be bonded to the inner surface of the scaffold 102 along a plurality of bond lines 802. All other portions of the membrane 104 remain uncoupled or unattached to the stent scaffold 102. This creates areas where the membrane 104 is separated from the stent scaffold 102 by a gap 204. Because the membrane 104 is not attached to the stent scaffold at all locations, the membrane 104 is free to flex inward and outward in response to changes in the fluid flow and/or pressure through the lumen 108. For example, as shown in FIG. 4, at a first cross-sectional location along the length of the lumen 108, there may be gaps 204 between the stent scaffold 102 and the membrane 104 between point locations 206 where the stent scaffold 102 and the membrane 104 are directly coupled together. Referring now to FIG. 5, in response to an increase in pressure within the lumen 108, the membrane 104 may flex outwardly towards the stent scaffold 102 reducing the gaps 204. Alternatively, in response to increased flow velocity within the lumen 108, the membrane 104, may flex inwardly away from the stent scaffold 102 by virtue of Bernoulli's principle, thereby increasing the “rifling effect” and encouraging increased laminar flow when flow velocity is increased.

These and other embodiments for methods of creating vortical flow through a lumen may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The technology has been described with reference to specific exemplary embodiments. Various modifications and changes, however, may be made without departing from the scope of the present technology. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present technology and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology, as expressed in the following claims.

1. A stent device, comprising:
a stent scaffold having a first end and a second end; and
a membrane disposed along an inner surface of the stent scaffold forming a lumen between the first and second ends, wherein an inner surface of the lumen is configured to encourage vortical flow between the first and second ends.

2. A graft device according to claim 1, wherein the inner surface of the lumen comprises a helical pattern between the first and second ends.

3. A graft device according to claim 2, wherein:
the membrane comprises a plurality of linear membrane components coupled together along their side edges to form a plurality of ridges; and
the plurality of ridges extend between the first and second ends in a helical fashion.

4. A graft device according to claim 3, wherein:
a first edge of a first linear membrane component is thermally bonded to an edge of an adjacent second linear membrane component forming a first ridge; and
a second edge of the first linear membrane component is thermally bonded to an edge of an adjacent third linear membrane component forming a second ridge.

5. A graft device according to claim 2, wherein:
an outer surface of the membrane is bonded to the inner surface of the stent scaffold along a plurality of bond lines arranged in a helical manner between the first and second ends; and
the plurality of bond lines form a series of ridges forming the helical pattern between the first and second ends.

6. A stent device according to claim 1, wherein the membrane comprises polytetrafluoroethylene.

7. A stent device according to claim 1, wherein the stent scaffold comprises nitinol.

8. A stent device according to claim 1, wherein the membrane is:
attached to the stent scaffold at a plurality of point locations located along the inner surface of the stent scaffold; and
unattached from the stent scaffold at all other locations along the inner surface of the stent scaffold, wherein the unattached portion of the membrane is configured to flex with respect to the stent scaffold in response to changes in a fluid flow velocity and pressure through the lumen.

9. A deployable stent, comprising:
a stent scaffold having a first end and a second end, wherein the stent scaffold is configured to expand outwardly from a constrained first position to a second deployed position, wherein a diameter of the stent scaffold in the deployed position is greater than that of the constrained first position; and
a flexible membrane disposed along an inner surface of the stent scaffold forming a lumen between the first and second ends, wherein:
an inner surface of the lumen comprises a helical pattern extending between the first and second ends; and
the flexible membrane is configured to expand outwardly with the stent scaffold to the deployed position.

10. A deployable stent according to claim 9, wherein:
the flexible membrane comprises a plurality of linear membrane components coupled together along their side edges to form a plurality of ridges; and
the plurality of ridges extend between the first and second ends in a helical fashion.

11. A deployable stent according to claim 10, wherein:
a first edge of a first linear membrane component is thermally bonded to an edge of an adjacent second linear membrane component forming a first ridge; and
a second edge of the first linear membrane component is thermally bonded to an edge of an adjacent third linear membrane component forming a second ridge.

12. A deployable stent according to claim 9, wherein:
an outer surface of the flexible membrane is bonded to the inner surface of the stent scaffold along a plurality of bond lines arranged in a helical manner between the first and second ends; and
the plurality of bond lines form a series of ridges forming the helical pattern between the first and second ends.

13. A deployable stent according to claim 9, wherein the flexible membrane comprises polytetrafluoroethylene.

14. A deployable stent according to claim 9, wherein the flexible membrane is:
attached to the stent scaffold at a plurality of locations located along the inner surface of the stent scaffold; and
unattached from the stent scaffold at all other locations along the inner surface of the stent scaffold, wherein the uncoupled portion of the flexible membrane is configured to flex relative to the stent scaffold in response to changes in a fluid flow velocity and pressure through the lumen.

15. A method of forming a stent, comprising:
forming a lumen along an inner surface of a tubular shaped scaffold, wherein the lumen comprises a membrane having an inner surface with a helical pattern extending between a first end and a second end of the lumen.

16. A method of forming a stent according to claim 15, wherein forming a lumen comprises:
bonding a plurality of linear membrane components together along their side edges; and
arranging the plurality of linear membrane components in a longitudinal-linear-helical fashion between the first end and the second end of the lumen.

17. A method of forming a stent according to claim 16, wherein:
a first side edge of a first linear membrane component is thermally bonded to a side edge of an adjacent second linear membrane component; and
a second side edge of the first linear membrane component is thermally bonded to a side edge of an adjacent third linear membrane component.

18. A method of forming a stent according to claim 17, wherein each thermally bonded side edge forms a ridge between adjacent linear membrane components that extends from the first end to the second end of the lumen in a helical manner.

19. A graft device according to claim 15, wherein:
an outer surface of the membrane is bonded to the inner surface of the scaffold along a plurality of bond lines arranged in a helical manner between the first and second ends of the lumen; and
the plurality of bond lines form a series of ridges forming the helical pattern between the first and second ends.

20. A method of forming a stent according to claim 15, further comprising:
attaching the membrane to the scaffold at a plurality of locations located along the inner surface of the scaffold; and
leaving the membrane unattached to the scaffold at all other locations along the inner surface of the stent scaffold, wherein the unattached portion of the membrane is configured to flex outwardly towards the inner surface of the scaffold in response to changes in a fluid flow velocity through the lumen.
